(19) United States
(12) Patent Application Publication
(10) Pub. No.: US 2021/0123002 A1
(43) Pub. Date: Apr. 29, 2021

(54) APPETIZING AND SUMMER HEAT RELIEVING TANGXIN RAW CIDER AND HIGH-EFFICIENCY DEEP PROCESSING TECHNOLOGY THEREOF

(71) Applicants: SDIC ZHONGLU FRUIT JUICE CO., LTD., Beijing (CN); RUSHAN ZHONGCHENG FRUIT JUICE BEVERAGE CO., LTD, Weihai (CN)

(72) Inventors: Jiming ZHANG, BEIJING (CN); Chuanzhu LENG, BEIJING (CN); Nan JIANG, WEIHAI (CN); Jiangchangmei LU, WEIHAI (CN); Sixin WANG, BEIJING (CN); Baoshuang TIAN, BEIJING (CN); Jiangming FAN, BEIJING (CN); Meijun DU, BEIJING (CN); Xihong LI, BEIJING (CN); Luyin WANG, BEIJING (CN); Yi ZHU, BEIJING (CN); Chunhui HOU, BEIJING (CN); Genlin GUO, BEIJING (CN)

(21) Appl. No.: 16/858,622

(22) Filed: Apr. 25, 2020

(30) Foreign Application Priority Data
Oct. 25, 2019 (CN) 201911020694.4

(51) Int. Cl. C12G 3/025; C12H 1/00; C12H 1/052; C12H 1/22

(52) U.S. Cl. CPC C12G 3/025; C12H 1/22; C12H 1/0416; C12H 1/006

(57) ABSTRACT

The disclosure relates to an appetizing and summer heat relieving tangxin raw cider and a high-efficiency deep processing technology thereof. The high-efficiency deep processing technology comprises the following steps: (1) preparing raw pulp; (2) detoxifying patulin; (3) inoculating and precisely fermenting; (4) performing enzymolysis of residues; (5) clarifying and filtering; (6) ageing; and (7) canning after adsorbing and clarifying. According to the disclosure, aiming at a phenomenon that cider is muddy after being stored, it is solved by adsorption with a multi-stimuli responsive nano fiber modified resin. Exterior stimuli include change in environment temperature or acidity-basicity (pH value), light, electric field, magnetic field or specific molecules. In response to exterior stimuli, nano fiber may include physicochemical properties such as size, hydrophilicity and hydrophobicity, refractive index, transmittance, mechanical strength, color or conductivity.

### CROSS-REFERENCE TO RELATED APPLICATIONS

This application claims priority to Chinese Patent Application No. 201911020694.4 with a filing date of Oct. 25, 2019. The content of the aforementioned applications, including any intervening amendments thereto, are incorporated herein by reference.

### TECHNICAL FIELD

The disclosure belongs to the technical field of fermentation, and relates to an appetizing and summer heat relieving tangxin raw cider and a high-efficiency deep processing technology thereof.

### BACKGROUND OF THE PRESENT INVENTION

Cider is a low-alcohol strength fermented fruit wine produced from apple or apple juice, and is the second largest fruit wine in the world next only to wine. Cider has high nutritional ingredients and healthcare value, contains dual nutritional ingredients produced by fermentation of apple and organisms, amino acids required by human body and special fruit acids specific to cider, and can assist in human metabolism and balance maintenance. Apple also contains many minerals such as calcium and magnesium, which can help digestion and absorption of human, maintenance of acid-alkaline balance and control of in vivo balance.

The production of cider in China is just in its infancy, and it mainly adopts a fruit wine production process. The quality of the produced cider is affected by many factors such as varieties of apple, fermentation strains and fermentation conditions. In the processes of fermentation and storage, selection and purification of strains are the key factors affecting the yield, quality and flavor of cider. How to improve the fermentation yield on the basis of maintaining good flavor and endowing cider with special functional characteristics is a hot trend in the development of cider industry.

According to retrieval, patent documents related to this application are found, and specific disclosed contents are as follows:

Semi-sweet cider ZL201610372268.7 discloses a semi-sweet cider which is prepared by mixing and juicing different varieties of apple according to a certain proportion, and adding sour papaya juice, optimizing and performing expanding propagation on the apple skin with wild yeast, and then mixing and fermenting. Its advantages are as follows: a semi-sweet cider with good export feeling, strong fruit flavor and unique flavor is produced by utilizing the variable temperature mixed fermentation technology. The disadvantages are as follows: (1) culture and fermentation is performed utilizing wild bacteria on the surface of apple, with low strain purity degree and low fermentation yield; (2) cider is brewed using selected apple as a raw material, the utilization rate of raw material is low, young fruits and remnant fruits can also be used to brew cider with the same quality, but there are hidden dangers of the harm of patulin toxin hazard; (3) the color, taste and nutritional value of the liquor produced by fermentation are not rich, it is difficult to meet the special needs of different crowds.

2. ZL 201610532954 discloses a preparation method of low-alcohol cider. The alcohol strength of the cider prepared by using this method is 6.35%, which reaches the standard of low alcohol. The advantages of this method are as follows: the prepared cider prepared contains low alcohol accuracy, and after the process optimization, the produced cider is rich in aroma components, soft in taste, mellow and harmonious in body. The disadvantages are as follows: (1) addition of potassium sulfite to the wine can prevent browning and clarify, and the ability of regulation and control is limited, and turbidity will still appear in the later stage of storage; (2) addition of glucose oxidase can realize low alcohol fermentation, but lose glucose, and reduce the fermentation yield of the wine; (3) the nutritional function of the wine has not been strengthened and effectively developed.

3. A cider and its brewing process ZL 201610121188.4 discloses a rapid fermentation and high clarity cider brewing process, which has the advantages of simple brewing process, short fermentation time, high clarity, good taste and no headache after drinking. The disadvantages are as follows: (1) the utilization of raw materials is low, and the fermentation yield is low; (2) in the fermentation process, addition of liquor to mix liquor precision will affect the original pure flavor of cider, and reduce the nutritional value; (3) the function of cider is single, and the functionality of cider needs to be developed and strengthened.

Based on the above patents, it is found that the existing cider fermentation technology has the following problems:

1. Low fermentation yield

The main reason is that if wild strains are only used for fermentation, the purification degree is low; if artificial yeast is only used for fermentation, specificity is poor, the utilization rate of the substrate is low, and the fermentation conditions cannot achieve precise regulation.

Solution: yeast strains with high specificity are selected from orchard soil, mixed with wild yeast of apple skin, decompression temperature-variable and nitrogen-disturbance precise regulation manner is utilized for fermentation to enhance the vitality and fermentation ability of the strains. Cellulase and pectinase are added in the filtered residue, and the decomposed products continue to be circularly fermented, thereby improving the utilization rate and yield of raw materials.

2. Selected fruits are used for brewing, with limited raw material utilization range. Young fruits and broken fruits can brew cider having the same quality, but there are hidden dangers of patulin in the brewed cider.

In order to improve the taste and safety of the product, the traditional technology mostly uses the selected apple as the raw material to brew cider. However, the fruits after apple blossom drop and the artificially removed young fruits can also brew cider having the same value, but there is the safety hidden danger caused by cyanogenase toxin residue in the cider.

3. The functionality of fruit wine is single, and the functionality of clearing heat and refreshing has not been strengthened and effectively developed.

The main reason is that the production of the most fruit wines starts from the taste, flavor and original purity, rarely considers the consumption needs of special people, so the cider having the function of reliving summer hear has not been fully developed. Cider is warm and refreshing, has cool and refreshing feel after drinking. As long as ingredients are slightly conditioned, it can become a good product for clearing heat to refresh.

4. Fruit wine will be turbid or precipitated after long-term storage

It is mainly biological turbidity and non biological turbidity. Biological turbidity is haze, turbidity and precipitate produced due to the destruction of the colloidal balance of fruit wine by microorganisms. Non-biological turbidity is separated out by aggregation of biological macromolecules such as fermented fruit wine deterioration, cellulose and tannin under a series of physical and chemical conditions.

### SUMMARY OF PRESENT INVENTION

The object of the disclosure is to provide a strengthened fermentation processing technology of high-yield cider having a function of clearing heat to refresh and solve the key technical problems that high-yield strains are selected to prevent post turbidity.

The technical solution to achieve the object of the disclosure is as follows:

An appetizing and summer heat relieving tangxin raw cider, comprising the following steps:

(1) preparing raw pulp:

selecting apple fruits after flowers drop, artificially removing young fruits, getting rid of mildew parts, washing and removing surface dirts, drying, slitting, removing kernels, pulping in a ratio of water to fruit of 8:5, simultaneously adding 1.2% Vc and 0.05% NaCl to protect color and prevent browning; then culturing in a 25° C. incubator, performing expanding propagation on skin with wild yeast for 24 hours, sucking 1 mL of fermentation broth every 2 h and diluting the fermentation broth with distilled water into 30 times of liquid, and transferring the diluted fermentation broth to 0-3° C. refrigerator for later use when the total number of yeast in each ml of fermentation liquid reaches 10⁴ order of magnitude;

(2) detoxifying patulin

biologically detoxifying the apple pulp: inoculating plant lactic acid bacteria (ATCC 8014) into the pulp, stirring and detoxifying for 20-24 h at 20-30° C. and under the condition of adjusting pH 3-7, and then adsorbing and filtering with a carboxylated nano multi-wall carbon-neutral aluminum oxide filter sieve plate for three times;

(3) inoculating and precise fermentation:

inoculating Saccharomyces cerevisiae (ATCC 9080) into the raw pulp amplified by wild yeast in an inoculation amount of 8-12%, and precisely controlling decompression and variable temperature fermentation, wherein the pressure of a decompression fermentation tank is 0.08±0.01 Mpa, the temperature-variable fermentation manner is as follows: the temperature is controlled at 25±0.05° C. on the first 1-2 days of fermentation, the temperature is controlled at 15±0.05° C. on the days 3-5 of fermentation, and the temperature is controlled at 20±0.05° C. on the days 6-10 of fermentation, ending main fermentation when the sugar content dropped to be below 0.5%, feeding the supernatant into a closed fermentation barrel for post-fermentation during which nitrogen is filled every two days for disturbance fermentation of liquor, maintain the normal temperature and low pressure of 0.08±0.01 MPa, and fermenting for 10-15 d after normal temperature and low pressure of 0.08±0.01 MPa are maintained to obtain the raw wine;

(4) performing enzymolysis of residues:

adding 50 mg/L-70 mg/L of pectinase and 85 mg/L-100 mg/L of cellulase to the filtration residues, stirring for 24 h, and decomposing pectin and cellulose in the residues into monosaccharide and oligosaccharide for later cyclic fermentation;

(5) clarifying and filtering:

filtering using a clarifier which includes one or several mixtures of bentonite, gelatin and chitosan, with the addition amount of 1.5 g/L-2.5 g/L;

(6) ageing:

after residual wine in blended liquor with a siphoning method, ageing at 18-20° C. for 45-60 days, during which, on the 30th day, changing the fermentation barrel once and sealing for preservation;

(7) adsorbing and clarifying: adsorbing with multi-stimuli responsive nanofiber modified resin; and

(8) aseptic canning:

pasteurizing the adsorbed and clarified liquor for 20-30 min at 68-70° C., and then performing aseptic canning to obtain the finished product.

Furthermore, the structure of the filter screen plate is as follows: the filter screen has five layers of structures, the first layer is a sieve plate with a pore size of 5-10 meshes; the second layer is carboxylated nano multi-wall carbon with a particle size of 3-5 nm; the third layer is macroporous adsorption resin; the fourth layer is neutral aluminum oxide with a particle size of 50-70 μm; the fifth layer is a sieve plate with a pore size of 5-10 meshes; the pulp containing solute with a particle size of less than 3 mm can pass through the adsorption filter screen plate.

Furthermore, 3-10% of Chinese herbal medicine extract having a function of clearing heat and relieving summer heat is added in the wine, and the preparation process of the extract is as follows:

the Chinese herbal medicine having a function of heat clearing and reliving summer heat comprises the following ingredients: 1-2 parts of mint, 2-3 parts of wild chrysanthemum, 1-2 parts of red cortex, 1-2 parts of red peony, 1-2 parts of Andrographis paniculata, 2-3 parts of liquorice, 1-2 parts of isatis root, 1-2 parts of Indigo naturalis, 1.5 parts of Smilax glabra, 1-2 parts of wild buckwheat, 1-2 parts of purslane, 2-3 parts of honeysuckles;

the main technical points of decompression ultrasonic assisted enzymatic extraction comprises: extraction pressure of 0.05-0.08 mpa, 2% complex enzyme, ultrasonic power of 165 W, ultrasonic temperature of 40° C., and twice ultrasonic extraction, 30 min each time.

Furthermore, the preparation method of multi-stimuli responsive nano fiber modified resin is as follows:

the multi-stimuli responsive nano fiber modified resin is prepared by using a polymer having multi-stimuli response and through an electrospinning process, specifically, by copolymerizing multiple monomers, comprising photosensitizer nanorod (AuNRs), temeperature-response N-isoproply acrylamide (NIPAM), 4-acrylaminobenzo-15-crown ether 5 group (having recognition response to glucose molecule), Fe3O4 nano particles (magnetic response), azobenzene modified polylactone (PCL), for synthesizing different stimuli-response polymers to form a copolymer, then adding PVP powder with absolute ethyl alcohol as a solvent to be prepared into 30% PVP solution, adding 5%-10% of copolymer to form spinning solution, stirring and dissolving at 20° C. until homogenous solution is formed, standing for 30 min, then adding to a pouring device of an electrostatic spinning apparatus for electrostatic spinning, wherein the pressure is set as 20 KV, a distance between a nozzle and a reception plate is 10 cm, liquid supply speed is 0.1 mm/min, and a reception screen is ion exchange resin.

The disclosure has the advantages and beneficial effects:

The disclosure adopts yeast mixed fermentation, decompression temperature-variable and nitrogen disturbance precise regulation manner is used for fermentation, so as to improve the efficiency and yield of fermentation; decompression shortens fermentation time, temperature variation improves the strong degree and fermentation ability of strains, nitrogen disturbance at regular time increases a contact area between the strains and a substrate, and improves the efficiency of fermentation; cooperative promotion of many manners greatly improves the efficiency and yield of fermentation.

2. For a fact that there is a potential safety hazard of patulin because young fruits and residual fruits are used to brew cider. The prepared fruit pulp is subjected to repeated filtration and adsorption with a plant lactobacillus biological detoxification combined with carboxylation nano multi-wall carbon and neutral aluminum oxide filter screen, so as to realize the efficient removal of patulin. The toxin residue is far lower than patulin toxin limit sanitary standard (GB14974-1994) of patulin in apple and hawthorn products in China (GB14974-1994).

3. Aiming at factors that cider has the single function, the functionality of clearing heat to refresh has not been enhanced and developed, the pharmacological components of cider are blended, the medical components are quickly and efficiently integrated into cider by a reduced pressure ultrasonic assisted enzymatic extraction technology so as to prepare functional cider whose function of clearing heat to refresh is strengthened.

4. Aims at the turbidity phenomenon in the cider in the later period of storage, it can be solved by adopting the multi-stimuli responsive nano fiber modified resin adsorption. A layer of polyvinylpyrrolidone (PVPP) nanofiber having multi-stimuli response is subjected to electrospinning on the surface of the multi-stage adsorption resin by using the electrospinning technology to produce a smart polymer fiber whose physical and chemical properties are changed in response to external stimuli and which has the advantages of large specific surface area, high porosity and fast response to external stimuli. PVPP is a non-toxic non-irritant, safe and stable polymer with strong selective adsorption capacity, it can be precipitated through the formation of hydrogen bond complex between carbonyl and polyphenols such as anthocyanin, flavonoid polyhydroxy derivatives, so as to reduce the phenols in fruit wine to achieve the effect of stabilizing the color of fruit wine. External stimuli include changes in ambient temperature or acidity-alkalinity pH, light, electric field, magnetic field or specific molecules. The physical and chemical properties of nanofibers that change in response to external stimuli may include size, hydrophobicity, refractive index, transmittance, mechanical strength, color or conductivity.

### DESCRIPTION OF THE DRAWINGS

FIG. 1 is a picture of cider prepared according to the disclosure.

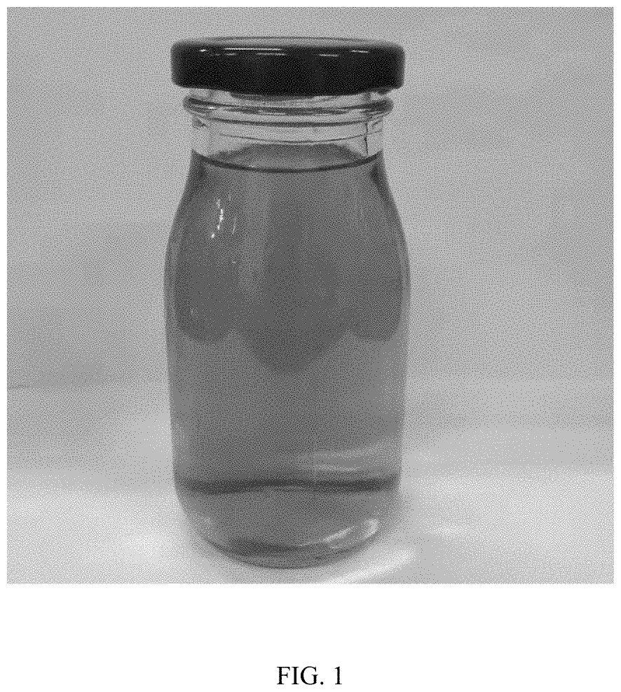

### DETAILED DESCRIPTION OF PREFERRED EMBODIMENTS

Next, the disclosure will be further described in detail, and the following examples are only illustrative but not limiting, and cannot hereby limit the protective scope of the disclosure.

An appetizing and summer heat relieving tangxin raw cider comprises the following steps:

(1) Preparing Raw Pulp:

selecting apple fruits after flowers drop, artificially removing young fruits, getting rid of mildew parts, washing and removing surface dirt, drying, slitting, removing kernels, pulping in a ratio of water to fruit of 8:5, simultaneously adding 1.2% Vc and 0.05% NaCl to protect color and prevent browning; then culturing in a 25° C. incubator, performing expanding propagation on the skin with wild yeast for 24 hours, sucking 1 mL of fermentation broth every 2 h and diluting the fermentation broth with distilled water into 30 times of liquid, transferring the diluted fermentation broth to 0-3° C. refrigerator for later use when the total number of yeast in each ml of fermentation liquid reaches 10⁴ order of magnitude;

(2) Patulin Detoxifying Treatment:

biologically detoxifying the apple pulp: inoculating plant lactic acid bacteria (ATCC 8014) into the pulp, stirring and detoxifying for 20-24 h at 20-30V and under the condition of adjusting pH 3-7, and then adsorbing and filtering with a carboxylated nano multi-wall carbon-neutral aluminum oxide filter sieve plate for three times. Detoxification effect (detection of detoxification residues) is as shown in Table 1.

The structure of the filter screen plate is as follows: the filter screen has five layers of structures, the first layer is a sieve plate with a pore size of 5-10 meshes; the second layer is carboxylated nano multi-wall carbon with a particle size of 3-5 nm; the third layer is macroporous adsorption resin; the fourth layer is neutral aluminum oxide with a particle size of 50-70 μm; the fifth layer is sieve plate with a pore size of 5-10 meshes; the pulp containing solute with a particle size of less than 3 mm can pass through the adsorption filter screen plate.

(3) Inoculating and Precise Fermentation:

inoculating Saccharomyces cerevisiae (ATCC 9080) into the raw pulp amplified by wild yeast in an inoculation amount of 8-12%, and precisely controlling decompression and variable temperature fermentation, wherein the pressure of a decompression fermentation tank is 0.08±0.01 Mpa, the temperature-variable fermentation manner is as follows: the temperature is controlled at 25±0.05° C. on the first 1-2 days of fermentation, the temperature is controlled at 15±0.05° C. on the days 3-5 of fermentation, and the temperature is controlled at 20±0.05° C. on the days 6-10 of fermentation, ending main fermentation when the sugar content dropped to be below 0.5%, feeding the supernatant into a closed fermentation barrel for post-fermentation during which nitrogen is filled every two days for disturbance fermentation of liquor, maintain the normal temperature and low pressure of 0.08±0.01 MPa, and fermenting for 10-15 d after normal temperature and low pressure of 0.08±0.01 MPa are maintained to obtain the raw wine;

(4) Performing Enzymolysis of Residues:

adding 50 mg/L-70 mg/L of pectinase and 85 mg/L-100 mg/L of cellulase to the filtration residues, stirring for 24 h, and decomposing pectin and cellulose in the residues into monosaccharide and oligosaccharide for later cyclic fermentation;

(5) Clarifying and Filtering:

filtering using a clarifier which includes one or several mixtures of bentonite, gelatin and chitosan, with the addition amount of 1.5 g/L-2.5 g/L.

(6) Blending Ingredients:

the Chinese herbal medicine having a function of heat clearing to refresh comprises the following ingredients: 1-2 parts of mint, 2-3 parts of wild chrysanthemum, 1-2 parts of red cortex, 1-2 parts of red peony, 1-2 parts of Andrographis paniculata, 2-3 parts of liquorice, 1-2 parts of isatis root, 1-2 parts of Indigo naturalis, 1.5 parts of Smilax glabra, 1-2 parts of wild buckwheat, 1-2 parts of purslane, and 2-3 parts of honeysuckles.

The main technical points of decompression ultrasonic assisted enzymatic extraction comprises: extraction pressure of 0.05-0.08 mpa, 2% complex enzyme, ultrasonic power of 165 W, ultrasonic temperature of 40° C., and twice ultrasonic extraction, 30 min each time.

(7) Ageing:

after residual wine in blended liquor with a siphoning method, ageing at 18-20° C. for 45-60 days, during which, on the 30th day, changing the fermentation barrel once and sealing for preservation.

(8) Adsorbing and Clarifying:

adsorbing with multi-stimuli responsive nanofiber modified resin. The preparation method of stimulus responsive PVPP electrospinning nano fiber is as follows: the multi-stimuli responsive nano fiber modified resin is prepared by using a polymer having multi-stimuli response as a fiber forming polymer and through an electrospinning process, specifically, by copolymerizing multiple monomers, comprising photosensitizer nanorod (AuNRs), temeperature-response N-isoproply acrylamide (NIPAM), 4-acrylaminobenzo-15-crown ether 5 group (having recognition response to glucose molecule), Fe3O4 nano particles (magnetic response), azobenzene modified polylactone (PCL), for synthesizing different stimuli-response polymers to form a copolymer, then adding PVP powder with absolute ethyl alcohol as a solvent to be prepared into 30% PVP solution, adding 5%-10% of copolymer to form spinning solution, stirring and dissolving at 20° C. until homogenous solution is formed, standing for 30 min, then adding to a pouring device of an electrostatic spinning apparatus for electrostatic spinning, wherein the pressure is set as 20 KV, a distance between a nozzle and a reception plate is 10 cm, liquid supply speed is 0.1 mm/min, and a reception screen is ion exchange resin.

The adsorption resin with specific stimulus response is prepared. The filtration flow rate of cider is 2 By/h, which ensure the daily average change rate of color value (dT440/d)<1.0. Loss of the most amino acid substances in cider treated by a single ion exchange resin is avoided. Polyvinylpyrrolidone (PVPP) nanofibers with stimulus response on the surface is adopted for multi-stage adsorption, thereby effectively removing the substances such as gum, protein, phenols, metal ions and tannins that lead to secondary precipitation.

(9) Aseptic Canning:

pasteurizing the adsorbed and clarified liquor for 20-30 min at 68-70° C., and then performing aseptic canning to obtain the finished product.

TABLE 1
Detection of residues in patulin toxin

| | | Detoxify | Carboxylated nano | Detoxifica- |
|---|---|---|---|---|
| | Apple | with plant | multi-wall carbon | tion with |
| | raw | lactic acid | and aluminum oxide | combination |
| Method | stock | bacteria | adsorption | of both |
| Detection | >75-90 | 4.6-5.7 | 24-33 | 0.9-1.5 |
| of residue | μg/kg | μg/kg | μg/kg | μg/kg |
| amount | | | | |

Note:
The sanitary standard for the limit of patulin in China (gb14974-1994) does not exceed 50 μg/kg.

TABLE 2
Nutrient ingredient list (content in 100 g of cider)

| | | | |
|---|---|---|---|
| Energy (kJ) | 531 | Protein (g) | 1.2 |
| Fat (g) | 0 | Saturated fatty acid | 0 |
| Carbohydrate (g) | 4.3 | Cholesterol (mg) | 0 |
| Dietary fiber (g) | 2.5 | Vitamin A (ug) | 2.7 |
| Carotene (ug) | 0.4 | Retinol (ug) | 57.3 |
| Sodium (mg) | 2 | Vitamin D (ug) | 0.1 |
| Thiamine (mg) | 0.05 | Riboflavin (mg) | 0.05 |
| Niacin (mg) | 0.5 | Vitamin C (mg) | 12 |
| Vitamin E (mg) | 3.23 | K (mg) | 121 |
| Ca (mg) | 7 | Mg (mg) | 6 |
| Fe (mg) | 0.9 | Mn (mg) | 0.01 |
| Zn (mg) | 0.21 | P (mg) | 9 |
| Se (mg) | 0.19 | F (mg) | 0 |

We claim:

1. A high-yield fermentation process of appetizing and summer heat relieving tangxin raw cider, comprising the following steps:
(1) preparing raw pulp
selecting apple fruits after flowers drop, artificially removing young fruits, getting rid of mildew parts, washing and removing surface dirts, drying, slitting, removing kernels, pulping in a ratio of water to fruit of 8:5, simultaneously adding 1.2% Vc and 0.05% NaCl to protect color and prevent browning; then culturing in a 25° C. incubator, performing expanding propagation on a skin with wild yeast for 24 hours, sticking 1 mL of fermentation broth every 2 h and diluting the fermentation broth with distilled water into 30 times of liquid, transferring the diluted fermentation broth to a 0-3° C. refrigerator for later use when the total number of yeast in each ml of fermentation liquid reaches 10⁴ order of magnitude;
(2) detoxifying patulin
biologically detoxifying the apple pulp: inoculating plant lactic acid bacteria (ATCC 8014) into the pulp, stirring and detoxifying for 20-24 h at 20-30° C. and under the condition of adjusting pH 3-7, and then adsorbing and filtering with a carboxylated nano multi-wall, carbon-neutral aluminum oxide filter screen plate for three times;
(3) inoculating and precisely fermenting:
inoculating Saccharomyces cerevisiae (ATCC) 9080) into the raw pulp amplified by wild yeast in an inoculation amount of 8-12%, and precisely controlling decompression and variable temperature fermentation, wherein the pressure of a decompression fermentation tank is 0.08±0.01 Mpa, the temperature-variable fermentation manner is as follows: the temperature is controlled at 25±0.05° C. on the first 1-2 days of fermentation, the temperature is controlled at 15±0.05° C. on the days 3-5 of fermentation, and the temperature is controlled at 20±0.05° C. on the days 6-10 of fermentation, ending main fermentation when the sugar content dropped to be below 0.5%, feeding the supernatant into a closed fermentation barrel for post-fermentation during which nitrogen is filled every two days for disturbance fermentation of liquor, maintaining the normal temperature and low pressure of 0.08±0.01 MPa, and fermenting for 10-15 d after normal temperature and low pressure of 0.08±0.01 MPa are maintained to obtain the raw wine;
(4) performing enzymolysis of residues:
adding 50 mg/L-70 mg/L of pectinase and 85 mg/L-100 mg/L of cellulase to the filtered residues, stirring for 24 h, and decomposing pectin and cellulose in the residues into monosaccharide and oligosaccharide for later cyclic fermentation;
(5) clarifying and filtering:
filtering using a clarifier which includes one or several mixtures of bentonite, gelatin and chitosan, with the addition amount of 1.5 g/L-2.5 g/L;
(6) ageing:
after residual wine in blended liquor is removed with a siphoning method, ageing, at 18-20° C. for 45-60 days, during which, on the 30th day, changing the fermentation barrel once and sealing for preservation;
(7) adsorbing and clarifying: adsorbing with multi-stimulus responsive nanofiber modified resin; and
(8) aseptic canning:
pasteurizing the adsorbed and clarified liquor for 20-30 min at 68-70° C., and then performing aseptic canning to obtain the finished product.

2. The appetizing and summer heat relieving tangxin raw cider according to claim 1, wherein the structure of the filter screen plate is as follows: the filter screen has five layers of structures, the first layer is a sieve plate with a pore size of 5-10 meshes; the second layer is carboxylated nano multi-wall carbon with a particle size of 3-5 nm; the third layer is macroporous adsorption resin; the fourth layer is neutral aluminum oxide with a particle size of 50-70 μm; the fifth layer is a sieve plate with a pore size of 5-10 meshes; the pulp containing solute with a particle size of less than 3 mm can pass through the adsorption filter screen plate.

3. An high-efficiency deep processing technology of appetizing and summer heat relieving tangxin raw cider according to claim 1, wherein 3-10% of Chinese herbal medicine extract having a function of clearing heat and reliving summer heat is added in the wine, and the preparation process of the extract is as follows:
the Chinese herbal medicine having a function of heat clearing and reliving summer heat comprises the following ingredients: 1-2 parts of mint, 2-3 parts of wild chrysanthemum, 1-2 parts of red cortex, 1-2 parts of red peony, 1-2 parts of Andrographis paniculata, 2-3 parts of liquorice, 1-2 parts of isatis root, 1-2 parts of Indigo naturalis, 1.5 parts of Smilax glabra, 1-2 parts of wild buckwheat, 1-2 parts of purslane, 2-3 parts of honeysuckles;
the main technical points of decompression ultrasonic assisted enzymatic extraction comprises: extraction pressure of 0.05-0.08 Mpa, 2% complex enzyme, ultrasonic power of 165 W, ultrasonic temperature of 40° C.′>, and twice ultrasonic extraction, 30 min each time.

4. The high-efficiency deep processing technology of appetizing and summer heat relieving tangxin raw cider according to claim 3, wherein the preparation method of multi-stimuli responsive nano fiber modified resin is as follows:
the multi-stimuli responsive nano fiber modified resin is prepared by using a polymer having multi-stimuli response and through an electrospinning process, specifically, by copolymerizing multiple monomers, comprising photosensitizer nanorod (AuNRs), temeperature-response N-isoproply acrylamide (NIPAM), 4-acrylaminobenzo-15-crown ether 5 group (having recognition response to glucose molecule), Fe3O4 nano particles (magnetic response), azobenzene modified polylactone (PCL), for synthesizing different stimuli-response polymers to form a copolymer, then adding PVP powder with absolute ethyl alcohol as a solvent to be prepared into 30% PVP solution, adding 5%-10% of copolymer to form spinning solution, stirring and dissolving at 20° C. until homogenous solution is formed, standing for 30 min, then adding to a pouring device of an electrostatic spinning apparatus for electrostatic spinning, wherein the pressure is set as 20 KV, a distance between a nozzle and a reception plate is 10 cm, the liquid supply speed is 0.1 mm/min, and a reception screen is ion exchange resin.
